(19) United States
(12) Patent Application Publication
(10) Pub. No.: US 2009/0293881 A1
(43) Pub. Date: Dec. 3, 2009

(54) MASK FOR OXYGEN DELIVERY WITH MEDICATION INLET

(76) Inventor: WILLIAM B. GRAHAM, St. Charles, MO (US)

Correspondence Address: CHARLES C. MCCLOSKEY, 763 S. NEW BALLAS ROAD STE. 170, ST. LOUIS, MO, 63141, US

(21) Appl. No.: 12/475,190

(22) Filed: May 29, 2009

Related U.S. Application Data
(60) Provisional application No. 61/057,524, filed on May 30, 2008.

(51) Int. Cl. A61M 16/06

(52) U.S. Cl. 128/207.12

(57) ABSTRACT

A mask delivers oxygen and medication to a patient simultaneously without lifting and inserting a second line beneath the mask. This mask has an extension with fittings for at least two functions, including oxygen delivery and medication delivery through a medication supply device. The extension has the fittings mutually proximate and generally adjacent in a compact space for minimal disruption to the patient and ease of inspection by the medical staff. The mask also includes check valves for inhalation regulation of the patient. Regarding placement upon a patient's face, the mask has a perimeter flange and a strap for securement upon the head of a patient.

### CROSS REFERENCE TO RELATED APPLICATION

This non-provisional application claims priority to the provisional application for patent Ser. No. 61/057,524 filed May 30, 2008 which is commonly owned by the same inventor.

### BACKGROUND OF THE INVENTION

The mask for oxygen delivery with medication inlet generally relates to breathing masks and more specifically to a mask with inlets for both breathing supply and delivery of gaseous and vaporous medication.

Upon receiving medical care, a patient may present with respiratory symptoms from a host of diseases and conditions. The patient may require treatment to reestablish or to strengthen breathing and to provide gaseous or vaporous medication into the lungs. Medical staff re-establishes and strengthen breathing using a variety of devices and techniques. The devices often share a mask that seals the nose and mouth of a patient for delivery of air, oxygen, or other gases into the respiratory system. One device is a non-rebreather that sustains a predetermined percentage of oxygen inhaled by the patient. During delivery of oxygen, many of these patients require further medication delivered via a medication supply device. A medication supply device provides a chamber that holds liquid or dissolved medication and that receives pressurized air or oxygen. The gas passing through the chamber collects medicinal vapors that pass through a line for inhalation by a patient, again through mask.

In emergency rooms in many cities, patients arrive with respiratory conditions ranging from asthma to pneumonia. Many patients require a non-rebreather to sustain their oxygen uptake for life support. Often, these patients enter an emergency room wearing a non-rebreather mask while suffering from an asthma attack, COPD, pneumonia, or a cardiac crisis. These conditions often call for medicinal treatment delivered by a medication supply device. However, the connection of a medication supply device to the respiratory system interrupts the non-rebreather operation which puts the patient at risk.

A non-rebreather mask operates upon 10 to 15 liters per minute oxygen supply while a medication supply device operates upon approximately 7 liters per minute of medicated oxygen flow. Though the patient requires the medication from the medication supply device, medical staff must maintain the patient's oxygen saturation at a safe level.

The present invention seeks a device to deliver nebulized, or aerosolized, medication simultaneous with oxygen and without interrupting or decreasing the oxygen flow to a patient wearing a non-rebreather mask. The present invention provides an inlet for aerosolized medication that avoids blocking visual inspection of the adjacent breathing supply inlet.

### DESCRIPTION OF THE PRIOR ART

Over the years, various masks and adapters have sought to perform both oxygen supply and aerosol medication delivery. The prior art has not yet perfected delivery of nebulized medication with oxygen supply in a non-rebreather mask. A non-rebreather has a mask connected by a check valve to a bag that serves as a reservoir of oxygen supplied to a patient.

One prior art device provides a Y shaped adaptor where one branch connects to a mask and the other branch connects to a reservoir bag while the stem connects to an oxygen supply.

In medical locations nationally, medical staff provide nebulized medication to patients while leaving the non-rebreather mask upon the patient. The staff closes one end of the t-piece with tape for single direction air flow then the t-piece is turned and slid between the mask and the face of the patient. However, insertion of the medication supply device line beneath the perimeter of the mask provides a poor seal upon the patient's face. Though this method delivers oxygen and nebulized medication simultaneous, the method induces discomfort to the patient and lowers the delivery rates for both oxygen and medication.

Another technique uses a medication supply device mask in conjunction with a non-rebreather mask. This method lifts the non-rebreather mask forward from the patient's nose, breaking the seal upon the patient's face, disrupting oxygen delivery and places a medication supply device mask beneath the aerosol mask. The disruption of one mask overlaying another causes the non-rebreather mask to function poorly.

A third method removes the non-rebreather mask, then uses a nasal cannula in conjunction with a medication supply device mask. This method involves changing from one oxygen delivery method to two separate delivery methods. This method may meet the patient's oxygen requirement but medical staff must remove the patient from oxygen to begin this method. Placed beneath the edge of the mask, the cannula breaks the facial seal of the mask while delivering oxygen. However, interruption or delay in oxygen delivery for some patients may risk their lives.

The U.S. Pat. No. 5,586,551 to Hilliard shows an oxygen mask with nebulizer. This mask has an extended nose region well beyond the tip of a patient's nose. This nose region admits two openings, generally spaced along a line extending outwardly from a patient's chin. One opening may block inspection of the other opening.

The present invention overcomes the difficulties of the prior art. That is, the prior art has utilized various masks for single purposes, oxygen delivery, or medication delivery. The prior art has also used various fittings and techniques where a mask for a single function undergoes rigging to serve multiple purposes often inducing discomfort in a patient and reducing the amount of oxygen delivered or medication delivered to the patient as well. The present invention has appropriate valves for simultaneous delivery of oxygen and nebulized medication to a patient without breaking the seal of the mask to the patient's face or interrupting oxygen delivery and rebreathing.

### SUMMARY OF THE INVENTION

Generally, the present invention is a mask that both delivers oxygen and medication to a patient simultaneously without obscuring the breathing supply. This mask has an extension with nearly side by side fittings for at least two functions, including breathing supply or oxygen delivery and medication delivery. The extension has the fittings collocated and generally side by side in a compact space proximate to the face of a patient for minimal disruption to the patient and ease of use by the medical staff. The mask also includes at least one check valve and a strap for securement upon the head of a patient.

There has thus been outlined, rather broadly, the more important features of the invention in order that the detailed description thereof that follows may be better understood and that the present contribution to the art may be better appreciated. The present invention also includes a check valve for medication delivery, an inlet for breathing supply line, a tilt of the second inlet for medication delivery away from the first inlet, a tilt of the extension to the left of a patient, and both inlets locating near each other and close to the cheeks of a patient during usage. Additional features of the invention will be described hereinafter and which will form the subject matter of the claims attached.

Numerous objects, features and advantages of the present invention will be readily apparent to those of ordinary skill in the art upon a reading of the following detailed description of the presently preferred, but nonetheless illustrative, embodiment of the present invention when taken in conjunction with the accompanying drawings. Before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, the phraseology and terminology employed herein are for the purpose of description and should not be regarded as limiting.

One object of the present invention is to provide a new and improved mask for delivery of breathing gases, including oxygen, with another inlet for supply of medication.

Another object is to provide such a mask that delivers both oxygen and aerosolized medication to a patient.

Another object is to provide such a mask that allows medical staff to inspect both the breathing supply and the medication delivery simultaneous with neither obscuring the other.

Another object is to provide such a mask that has a medication supply inlet at an angle to the breathing supply inlet.

Another object is to provide such a mask that incorporates various controls to assure the proper flow of breathing gases and medication and that backflows for either flow are minimized.

Another object is to provide such a mask that may be readily and efficiently manufactured and marketed at a price affordable to the suppliers of medical devices and eventually the patients.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated a preferred embodiment of the invention.

### BRIEF DESCRIPTION OF THE DRAWINGS

In referring to the drawings,

FIG. 1 illustrates a front view of the preferred embodiment of the present invention and auxiliary delivery lines;

FIG. 2 shows a top view of the invention looking downwardly from the nose of the patient; and,

FIG. 3 shows a side view of the invention.

The same reference numerals refer to the same parts throughout the various figures.

### DESCRIPTION OF THE PREFERRED EMBODIMENT

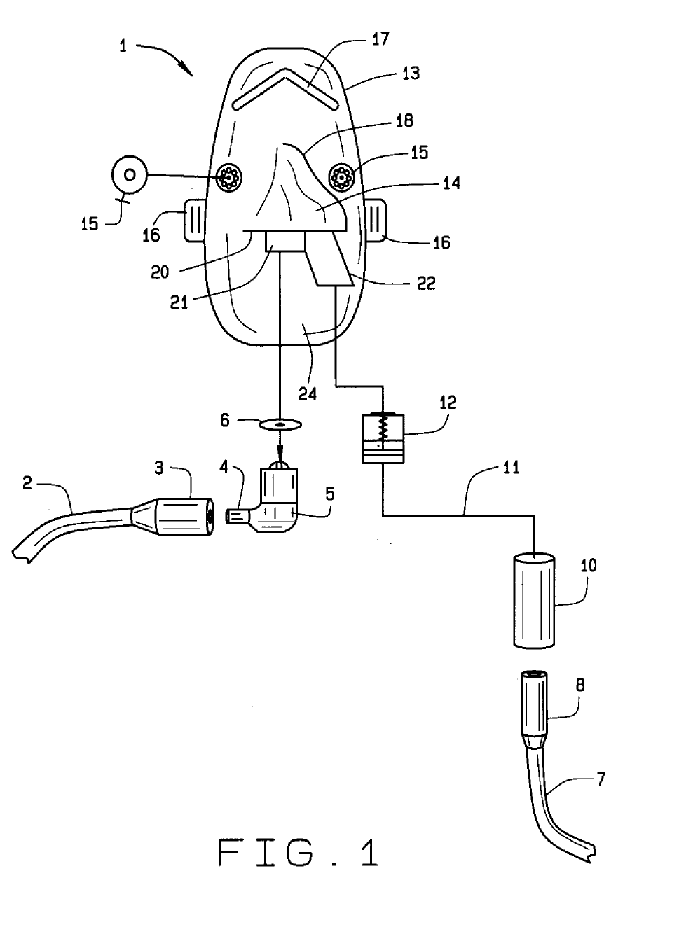

The present invention overcomes the prior art limitations and provides a mask for oxygen delivery simultaneously with nebulized medications. The preferred embodiment of the present invention 1 is shown in FIG. 1 in a partially exploded view with appurtenant oxygen supply lines. Breathing oxygen flows through a line 2, here shown on the left, to a fitting 3 generally of larger diameter than the line. The fitting, generally hollow, allows for continuous oxygen flow from the hollow line 2. The fitting has a central aperture that receives a tube 4 extending from an elbow 5 of a breathing supply line. The elbow delivers breathing gases, or oxygen, to a patient at a predetermined delivery rate. Alternatively, the present invention includes a check valve, as at 6, so that the breathing supply only opens into the mask 1 during inhalation by a patient. Additionally, oxygen also flows through a second line 7 and exits at a fitting 8 upon an end of the second line. The fitting delivers oxygen, also at a predetermined rate likely different from the other line 2, to a tube as at 7 depending from a medication supply device as at 10. The medication supply device can be a socket as shown or other device presently available for delivery of aerosolized medication. Here, the medication supply device admits oxygen through an aqueous solution in a transparent chamber thus vaporizing a medication for inhalation. The vaporized medication passes through a further line 11 that terminates at a biased one way valve 12, such as a check valve. The biased valve connects to an inlet also of the invention.

The invention provides a mask having special features as at 1. The mask has a generally concave body 13 having an elongated rectangular form with rounded corners. The body extends outwardly in an extension 14 from the remainder of the body. The extension has a length slightly more than the average nose length but is wider than the average nose width of a patient. The extension remains visually aesthetic and snug to the cheeks. The extension is generally located upon the mask at a location suitable for insertion of the nose of a patient while the remainder of the mask seals to the cheeks, chin, and upper face of the patient. Outwardly from the extension, the mask has at least one check valve as at 15. These check valves close when the mask attains a lower pressure as a patient inhales. The check valves then open when a patient exhales for exhausting carbon dioxide and other products of respiration. The check valves operate to maintain the presence of oxygen within the mask of the present invention. Outwardly from the check valves and generally at the level of the extension 14, the mask has slots for connection of the ends of a strap 16 as is known in the art. The strap has sufficient length or elasticity to reach around the head of a patient. The strap in cooperation with the material of the mask secures the mask upon the face of a patient in a tight seal. Above the extension, the mask has a spring strip 17, here shown as a chevron, that allows for bending of the mask to fit the face of a patient. The strip can attain the curvature of the upper part of a patient's nose or other facial feature for securement. Beneath the extension, the mask has a mouth cover 24 that extends from a maximum width near the strap and gently narrows towards where the chin of a patient abuts the mask during usage.

The extension has a generally hollow, broad, and partially triangular form slightly larger than the typical nose of a person, generally an adult though a pediatric version of this mask is foreseen. The extension has a bridge 18 generally extending at an angle from the plane of the mask and downwardly from the check valves 15. The extension curves from the bridge outwardly toward the body of the mask to the generally width of a patient's nose. The extension has a tip 19 generally located the farthest outward from the mask and also at the narrowest portion of the extension. From the tip and opposite the bridge, the extension has a base 20. The base is generally planar and flat, forming the bottom of the extension. The base extends perpendicular to the plane of the body of the mask, generally outwardly from the patient when wearing the present invention. The base has its narrow portion beneath the tip 19 and then expands in width towards the body 13 of the mask, of generally more width than a patient's nose. The base has its widest portion that generally locates beneath the nostrils of a patient adjacent to the upper lip.

The base also permits simultaneous connection of the breathing supply 2 through an optional check valve 6 and the further line 11 with its flow of medication through the biased valve 12 as part of the invention's mask. The check valve and the biased valve connect to the base through appropriate inlets that permit ready connection to the mask with limited interruption to oxygen and medication flow from the lines. The breathing supply to the base through a first inlet 21 depending from the base generally centered in the extension. The inlet has an inside diameter slightly less than the outside diameter of the check valve 6. The check valve fits snugly into the inlet in a friction, or press, fit that allows for ready connection and removal of the check valve. The check valve generally has a plastic tube with a nipple and a rubber one way valve, or disc, on one end that connects to the breathing supply on the other. The first inlet is a generally hollow cylindrical shape that opens beneath the center of the nostrils of a patient wearing the invention. A regular breathing supply has a plain nipple style adaptor that medical staff presses into the inlet for lower flow oxygen delivery. The inlet 21 can also accommodate a venturi style adaptor or other adaptors that fit the inlet and provide adequate oxygen flow to the patient.

The base also has a second inlet 22 here shown slightly outward and noticeably to the right of the first inlet 21. The second inlet has a longer length than the first inlet and as later shown, the second inlet is generally laterally adjacent to the first inlet. The second inlet has an outwardly angled orientation relative to the first inlet. The second inlet and the first inlet generally remain close to the face of the patient thus allowing medical staff to inspect the invention during usage without the medication supply blocking the breathing supply. The second inlet incorporates the biased valve 12 that admits the flow of aerosolized medication while preventing interruption of oxygen delivery. The second inlet is also a generally hollow cylindrical shape of slightly greater inside diameter than the outside diameter of the biased valve. The second inlet also provides for ready connection and removal of a check valve or direct medication supply device line connection. In an alternate embodiment, the first and second inlets each have similar inside diameters to permit connection of the check valve from the breathing supply to either inlet. Alternatively, the invention provides a cap for the second inlet that secondarily prevents leakage of oxygen through the second cap when a medication supply device is not used.

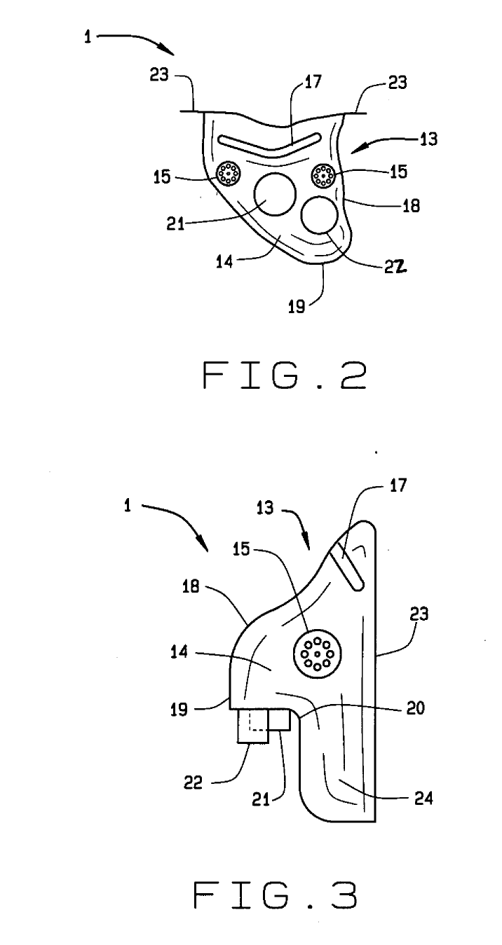

Turning the mask 1, FIG. 2 shows a top view of the present invention. As the mask is generally transparent, the first inlet and the second inlet appear below the bridge 18 in this figure. As before, the mask has a body 13 that covers a portion of the patient's face, generally including the nose and the mouth. The body has a perimeter flange, here shown divided as 23, that provides for a flush fit upon the skin of the patient's face. The body has the spring strip 17 generally locating towards the opening of the mask. Outwardly from the spring strip and slightly downward, the mask has two check valves 15, generally symmetric. Slightly forward of the check valves, the mask has the first inlet 21 here shown on end through the transparent bridge 18 and extension 14 of the invention. The first inlet is generally centered in the extension and upon the mask. Then the second inlet 22 is also shown on end though in front of the check valve on the left of a patient when wearing the mask. The second inlet locates to the side of the first inlet, here on the right of the figure and slightly more towards the tip 19 of the extension than the first inlet. The second inlet is approximately lateral to the first inlet. Both inlets approach the face of a patient during usage of the mask. The transparency of the mask allows viewing of the first and second inlet through the bridge though both inlets join with the base beneath the bridge and the extension.

The present invention appears from the side in FIG. 3. As before, the mask 1 has a body 13 with a mouth cover 24 towards the bottom and then the extension above the mouth cover. The extension 14 has a rounded, somewhat triangular shape in this view with the bridge 18 curving upwardly and inwardly to the body of the mask. The extension accommodates the average length nose but does not extend much further than that. The extension remains compact as the second inlet 22 is nearly lateral to the first inlet 21. The bridge continues upwardly and has a spring strip, as at 17, that aids in shaping the mask to fit the nose of a patient. Beneath the spring strip and away from the bridge, the mask has at least one check valve as at 15 that permits exhalation by a patient but not inhalation through the valve. Beneath the bridge, the extension widens to a base 20 that extends from the tip 19 inwardly to the remainder of the mask, generally the mouth cover 24. The base has a generally planar shape, mostly perpendicular to the remainder of the mask. The base has sufficient width, particularly in the direction across the mask, that is across the chin of a patient during usage, for the second inlet to locate nearly adjacent, that is, to the side of the first inlet. The base has a medication supply device inlet 22 proximate the tip and then a breathing supply inlet 21 inward and to the left of the medication supply device inlet. Behind the mouth cover and opposite the extension, the mask has a perimeter flange 23 that seals the mask to the face of a patient.

From the aforementioned description, a mask for oxygen delivery with medication inlet has been described. The mask is uniquely capable of delivering oxygen and aerosolized medication to a patient at the same time and without insertion of a tube beneath the flange of the mask. The mask further allows for prompt visual inspection of both breathing supply and medication supply line by medical staff during usage. The mask and its various components may be manufactured from many materials, including but not limited to, polymers, high density polyethylene, polypropylene, ferrous and non-ferrous metals, their alloys, and composites.

As such, those skilled in the art will appreciate that the conception, upon which this disclosure is based, may readily be utilized as a basis for the designing of other structures, methods and systems for carrying out the several purposes of the present invention. Therefore, the claims include such equivalent constructions insofar as they do not depart from the spirit and the scope of the present invention.

I claim:

1. A device aiding the respiration and treatment of a patient, the patient having a nose, cheeks, and a chin, comprising:
a mask having a body, generally elongated, an extension outwardly from said body slightly more in length than the nose of a patient, a mouth cover of said body beneath said nasal; and,
said extension adapting to connect to both a breathing supply line and a medication supply line at the same time.

2. The respiration and treatment aiding device of claim 1 further comprising:
said extension having a bridge generally linear and an opposite base generally planar and perpendicular to the plane of said mask, said base having a first inlet adapting to receive a breathing supply line, a second inlet, and a fitting connecting to said second inlet and said fitting adapting to receive a medication supply line, and said second inlet being nearly lateral and adjacent to said first inlet; and,
said extension adapting to remain close to the cheeks of a patient.

3. The respiration and treatment aiding device of claim 2 further comprising:
said second inlet having a greater length than said first inlet and locating at an angle to said first inlet wherein said device can be readily inspected during usage.

4. The respiration and treatment aiding device of claim 2 further comprising:
said first inlet having a greater width than said second inlet.

5. The respiration and treatment aiding device of claim 2 further comprising:
said fitting including a biased one way valve adapting to receive a medication supply line.

6. The respiration and treatment aiding device of claim 1 further comprising:
said mask being transparent and including at least one check valve upon said body, said at least one check valve preventing inflow of air into said mask during inhalation by a patient.

7. A device the respiratory treatment of a patient, the patient having a nose, cheeks, and a chin, comprising:
a mask having a body, generally elongated, an extension outwardly from said body slightly more in length than the nose of a patient, a mouth cover of said body beneath said extension;
said extension adapting to connect to both a breathing supply line and a medication supply line simultaneously;
said extension having a generally linear bridge and an opposite base generally planar and perpendicular to the plane of said mask, said base having a first inlet adapting to receive a breathing supply line, a second inlet, and a fitting in said second inlet, said fitting adapting to receive a medication supply line, and said second inlet being nearly lateral and adjacent to said first inlet; and,
said extension adapting to remain close to the cheeks of a patient;
wherein the breathing supply line connecting to said first inlet, the medication supply line connecting to said fitting of said second inlet, and said mask can be readily seen by medical staff.
